(19) United States
(12) Patent Application Publication
(10) Pub. No.: US 2007/0219451 A1
(43) Pub. Date: Sep. 20, 2007

(54) Optical Imaging Balloon Catheters

(76) Inventors: John Kula, Birdsboro, PA (US); Nicholas Green, Juniper, FL (US)

Correspondence Address: DIAMOND LAW OFFICE LLC, 1605 JOHN STREET, SUITE 102, FORT LEE, NJ, 07024, US

(21) Appl. No.: 11/681,312

(22) Filed: Mar. 2, 2007

Related U.S. Application Data
(60) Provisional application No. 60/778,393, filed on Mar. 3, 2006.

(51) Int. Cl. A61B 6/00; A61B 5/05; A61M 29/00

(52) U.S. Cl. 600/476; 600/407; 600/182; 604/96.01

(57) ABSTRACT

The present invention provides balloon catheters in which lateral-viewing optical components for interrogating the walls of blood vessel lumens are disposed within the lumen of the balloon(s). Advantageously, low pressure compliant balloons are used to minimize the possibility of injury to the blood vessels. The expanded balloons at least substantially clear blood from the optical path between the vessel wall that is interrogated and the lateral-viewing optics.

This application claims the benefit of U.S. provisional patent application Ser. No. 60/778,393 filed Mar. 3, 2006, which is incorporated by reference herein in its entirety.

### FIELD OF THE INVENTION

The invention relates generally to the field of vascular catheters having optical diagnostic capabilities.

### BACKGROUND OF INVENTION

Various optical modalities for diagnostically interrogating blood vessel walls to locate and characterize atherosclerotic lesions have been proposed. What is needed are improved catheter probes, particularly for the location and diagnosis of vulnerable plaque lesions. Vulnerable plaques, which are sometimes known as high-risk atherosclerotic plaques, include arterial atherosclerotic lesions characterized by a subluminal thrombotic lipid-rich pool of materials contained and/or overlaid by a thin fibrous cap. Although vulnerable plaques are non-stenotic or nominally stenotic, it is believed that their rupture, resulting in the release of thrombotic contents, accounts for a significant fraction of adverse cardiac events.

U.S. Pat. No. 4,976,710 discloses an angioscope employing a transparent balloon, and is incorporated by reference herein in its entirety.

U.S. Pat. No. 5,395,361 discloses an expandable fiber optic catheter for intraluminal laser transmission and, and is incorporated by reference herein in its entirety.

U.S. Pat. No. 5,415,654 discloses a laser balloon catheter apparatus for therapeutic treatment of prostate tissue, and is incorporated by reference herein in its entirety.

U.S. Pat. No. 6,701,181 discloses multi-path optical catheters, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,949,072 discloses devices for vulnerable plaque detection, and is incorporated by reference herein in its entirety.

U.S. Publication No. 2002/0193735 discloses helical angioplasty balloons for treating stenoses.

U.S. Publication No. 2002/0183622 discloses a fiber-optic apparatus and method for the optical imaging of tissue samples, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

U.S. Publication No. 2004/0204651 discloses infrared endoscopic balloon probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0075574 discloses devices for vulnerable plaque detection, and is incorporated by reference herein in its entirety.

### SUMMARY OF INVENTION

The present invention provides balloon catheters in which optical components for interrogating the walls of blood vessel lumens are disposed within the lumen of the balloon(s).

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIGS. 1A and 1B each show an embodiment of the invention in which lateral-viewing optics are disposed within an expandable (shown as expanded) low-pressure balloon near the distal end of an intravascular catheter.

FIG. 2 shows an embodiment of the invention in which a helical low-pressure balloon having lateral-viewing optics disposed within it is located near the distal end of an intravascular catheter.

FIG. 3 is a close-up view a helical balloon catheter embodiment of the invention showing the distal end of the catheter and a portion of the catheter proximal to the helical balloon.

### DETAILED DESCRIPTION

The present invention provides balloon catheters in which optical components for interrogating the walls of blood vessel lumens are disposed within the lumen of one or more inflatable balloon(s).

One embodiment of the invention provides an optical catheter for optically interrogating blood vessel walls that includes: an elongate catheter body having a distal end and a proximal end; a balloon section of the catheter comprising: a low-pressure inflatable balloon outwardly and radially disposed on the catheter, wherein the lumen of the balloon is in fluid communication with the proximal end of the catheter to permit inflation and deflation of the balloon; side-viewing optics disposed within the balloon section such that light can be emitted laterally through the balloon toward a vessel wall and light, such as light from the vessel wall, can be received for analysis.

Any suitable sort of side/lateral-viewing optical assembly(ies) may be used and numerous sorts of side-viewing optics are known in the art, for example a 45-deg (or other angle) mirror face or a prism can be used to laterally direct/redirect light from an optical fiber. Similarly an optical fiber can be provided with an angularly faceted tip to direct and receive light that is off-axis with respect to the fiber. In one embodiment, the optical balloon catheter includes least one optical fiber that longitudinally spans the body of the catheter from its proximal end to the side-viewing optics so that light transmitted from the proximal end of the catheter, such as from a laser, can be directed to the blood vessel wall and light received from the blood vessel wall can be transmitted out of the proximal end of the catheter for analysis. The lateral-viewing optical assembly(ies) may, for example, be part of or attached to a central shaft of the catheter.

In one embodiment of the invention, the balloon catheter includes at least two side-viewing optical assemblies, each having a different radial field of view. Each side-viewing optical assembly may be associated with at least one optical fiber that longitudinally spans the body of the catheter from its proximal end to the side-viewing optical assembly so that light transmitted from the proximal end of the catheter, such as from a laser, can be directed to the blood vessel wall and light received from the blood vessel wall can be transmitted out of the catheter for analysis. The side-viewing optics may, for example, include four side-viewing optical assemblies, each radially separated from adjacent assemblies by 90-deg or approximately 90-deg and each having an at least substantially non-overlapping radial field of view with the other. Each of these side viewing optical assemblies may be associated with at least one optical fiber that longitudinally spans the body of the catheter from its proximal end to the side-viewing optical assembly so that light transmitted from the proximal end of the catheter, such as from a laser, can be directed to the blood vessel wall and light received from the blood vessel wall can be transmitted out of the catheter for analysis.

The balloon may be a low-pressure balloon that is compliant so that the possibility of injury to a blood vessel, such as an interrogated artery is reduced and passage of the balloon through the blood vessel is facilitated. In one embodiment, the balloon is inflatable by 3 ATMs or less pressure. In a related embodiment, the balloon is inflatable by 2 ATMs or less pressure. In another embodiment, the balloon is inflatable by pressure between 1 ATM and 3 ATM. The balloon in its expanded state may have a circular profile transverse to the longitudinal axis of the catheter body. Thus, in its inflated state the balloon may, for example, have an ovaloid shape, a spherical shape, a cylindrical shape or a helical columnar shape.

In one embodiment, the balloon is helically shaped along the longitudinal axis of the catheter body, so that a helical groove permitting the passage of blood while the balloon is in its expanded state in a blood vessel extends from the proximal end of the balloon to the distal end of the balloon.

In a related embodiment, the balloon comprises a helically-shaped groove, channel or recess that extends from the proximal end of the balloon to the distal end of the balloon so that the passage of blood is permitted while the balloon is in an expanded state in a blood vessel. Other configurations are contemplated in accordance with the principles of the invention.

The balloon may be at least partially made of a transparent balloon material. The balloon need only be at least substantially transparent to the particular wavelengths of light that are required, to illuminate a target and receive back light for analysis, for a particular type of spectroscopy or optical analysis method. Thus, at least the part(s) of the balloon through which light is transmitted and received by the lateral-viewing optical assembly(ies) can be transparent to the extent required for a particular type of optical analysis. The balloon may also achieve a required level of transparency by having transparent windows through which light can pass that are not part of the general balloon material. Conventional balloon materials may, for example, be used, in accordance with the principles of the invention. Balloon materials may be selected according to the needs of a particular optical technique. Numerous polymers and polymer blends are available to select from. Suitable materials for the balloons of the invention may, for example, include polyethylenes (such as PE, HDPE and LDPE), polyesters (such as PET), nylons and polyamides generally, fluoropolymers (such as PTFE and FEP), silicones and polyurethanes.

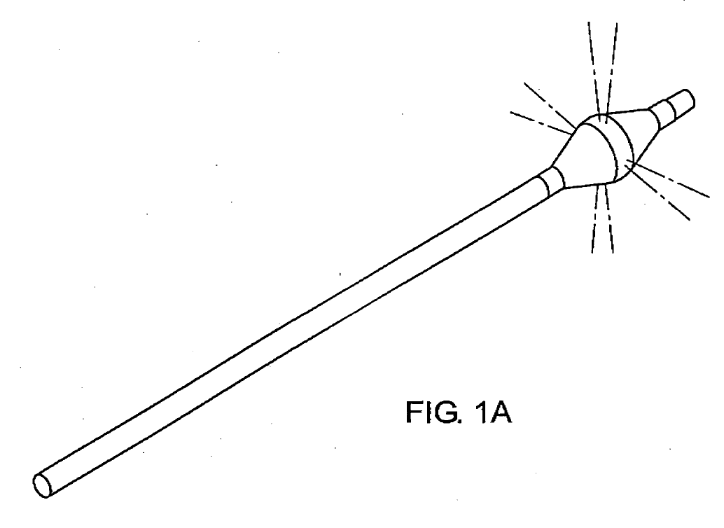

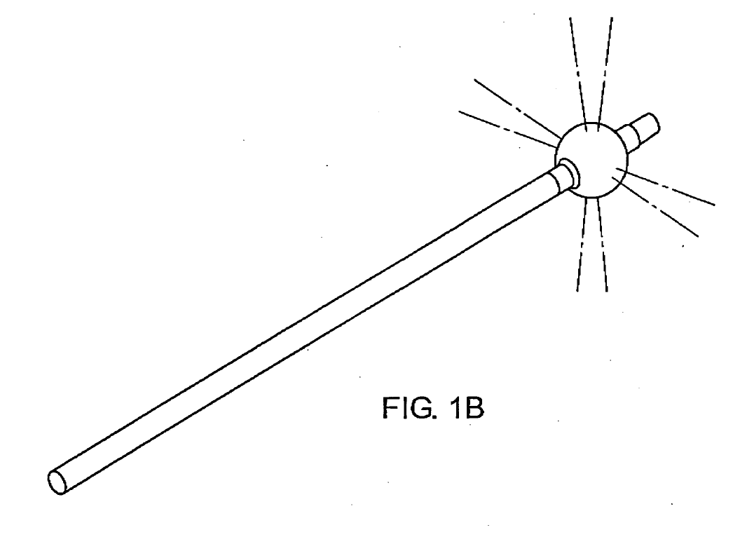

FIGS. 1A and 1B each show an embodiment of the invention in which lateral-viewing optics are disposed within an expandable (shown as expanded) low-pressure balloon near the distal end of an intravascular catheter. The balloons are shown in their expanded state. In their deflated state, the balloons conform closely to the catheter body. The four black columns shown extending from each balloon each indicate the field of view of one of four side-viewing optical assemblies that are present within the lumen of each balloon. The fields of view shown are non-overlapping and radially separated by about 90-degrees.

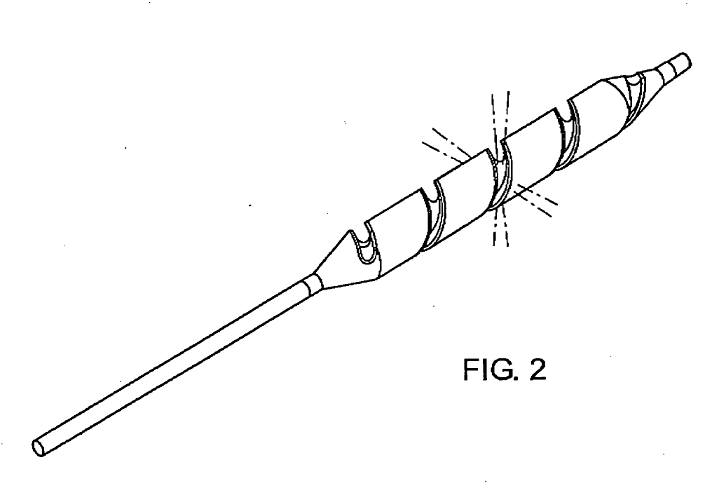

FIG. 2 shows an embodiment of the invention in which a helical low-pressure balloon having lateral-viewing optics disposed within it is located near the distal end of an intravascular catheter. The balloon presents a helical groove that extends from the proximal side of the balloon to the distal side of the balloon so that when the balloon is expanded in a blood vessel close to or even in intimate contact with the blood vessel wall, flowing blood can pass by the balloon by traveling through the helical groove. As in FIGS. 1A and 1B, the four black columns radiating from each balloon each indicate the field of view of one of four side-viewing optical assemblies that are present within the lumen of each balloon.

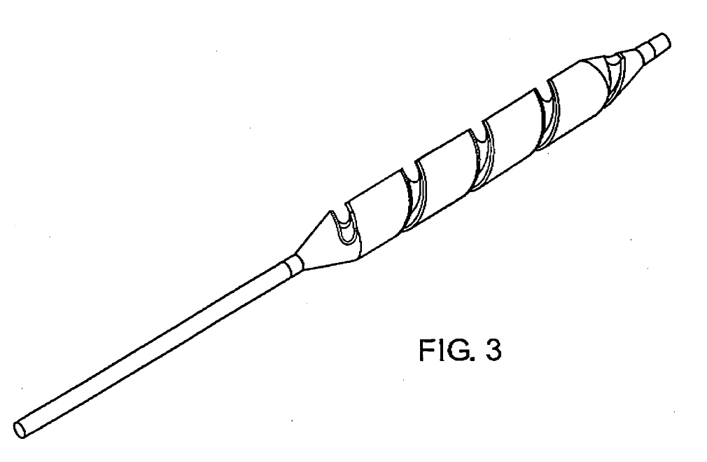

FIG. 3 is a close-up view a helical balloon catheter embodiment of the invention showing the distal end of the catheter and a portion of the catheter proximal to the helical balloon. The shaft of the catheter may have a conduit that extends through the catheter and out its tips for passage of a guidewire.

The balloon section of a catheter according to the invention, may for example, be located near the distal end of the catheter but not be part of the tip of the catheter, so that the distal end and proximal end of the balloon itself are attached to the body of the catheter. This particular configuration is shown in FIGS. 1A, 1B, 2 and 3.

An optical balloon catheter according to the invention may, for example, also comprise an inflation control technology so that a desired inflation diameter, for example, just below or at a specified distance below the lumen diameter of a blood vessel in which the balloon of the balloon catheter is present can be maintained. U.S. Pat. No. 6,706,004 and U.S. Publication No. 2002/0183620 described suitable systems, each of which is incorporated by reference herein in it entirety.

The invention provides methods for evaluating the condition of blood vessels using the optical catheter embodiments of the invention, which may, for example, include identifying, locating and/or characterizing atherosclerotic lesions in a blood vessel, such as an artery, using any of the optical catheter embodiments of the invention. A related embodiment of the invention provides methods for identifying, locating and/or characterizing vulnerable plaque lesions in a blood vessel, such as an artery, using any of the optical catheter embodiments of the invention. One embodiment of the invention is a method for diagnosing and/or locating one or more vulnerable plaque lesions in a blood vessel, such as a coronary artery of a subject, using a catheter as described herein to optically evaluate the properties of a vessel wall at one more locations along the vessel. Any of the methods of evaluating the condition of a blood vessel using an optical catheter according to the invention may include moving the catheter laterally within a blood vessel to optically interrogate the blood vessel wall at different lateral positions. Optical sampling of the vessel wall may be performed while the optical catheter is moving laterally within a blood vessel and/or while it is stopped at a lateral position within the vessel.

The invention is not limited by the optical method used to interrogate and diagnose the condition of a blood vessel wall. Suitable methods include, but are not limited to, low-resolution and high-resolution Raman spectroscopy, fluorescence spectroscopy, such as time-resolved laser-induced fluorescence spectroscopy, and laser speckle spectroscopy.

Differentially diagnosing and/or determining the location of a vulnerable plaque in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differentials characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Time-resolved laser-induced fluorescence methods for characterizing atherosclerotic lesions are disclosed in U.S. Pat. No. 6,272,376, which is incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated by reference herein in its entirety. A method and system for detecting and localizing vulnerable plaques based on the detection of biomarkers is disclosed in U.S. Pat. No. 6,860,851, which is hereby incorporated by reference herein in its entirety.

The invention also provides an integrated system for evaluating the status of a blood vessel wall, for example, for diagnosing and/or locating vulnerable plaque lesions, that includes an optical balloon catheter according to the invention, in communication with a light source such as a laser for illuminating a target region of a blood vessel via the catheter and a light analyzer, such as a spectroscope, for analyzing the properties of light received from the target region via the catheter. One or more computers, or computer processors generally working in conjunction with computer accessible memory under the control of computer instructions, e.g., software, may be part of the system for controlling the system and/or for analyzing information obtained by the system.

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. An optical catheter for optically interrogating blood vessel walls, comprising:
an elongate catheter body having a distal end and a proximal end;
a balloon section of the catheter comprising:
a low-pressure inflatable balloon outwardly and radially disposed on the catheter, wherein the lumen of the balloon is in fluid communication with the proximal end of the catheter to permit inflation and deflation of the balloon; and
side-viewing optics disposed within the balloon section such that light can be emitted laterally through the balloon toward a vessel wall and can be received for analysis.

2. The catheter of claim 1, wherein the balloon is inflatable by 3 ATMs or less pressure.

3. The catheter of claim 2, wherein the balloon is inflatable by 2 ATMs or less pressure.

4. The catheter of claim 1, wherein the balloon in its expanded state has a circular profile transverse to the longitudinal axis of the catheter body.

5. The catheter of claim 4, wherein the balloon is at least substantially sphere-shaped in its expanded state.

6. The catheter of claim 1, wherein the balloon is helically shaped along the longitudinal axis of the catheter body, so that a helical groove permitting the passage of blood while the balloon is in its expanded state in a blood vessel extends from the proximal end of the balloon to the distal end of the balloon.

7. The catheter of claim 1, wherein the balloon comprises a helically shaped groove that extends from the proximal end of the balloon to the distal end of the balloon so that the passage of blood is permitted while the balloon is in an expanded state in a blood vessel.

8. The catheter of claim 1, further comprising:
at least one optical fiber that longitudinally spans the body of the catheter from its proximal end to the side-viewing optics so that light transmitted from the proximal end of the catheter can be directed to a blood vessel wall and light received from a blood vessel wall can be transmitted out of the catheter for analysis.

9. The catheter of claim 1, wherein the side-viewing optics comprise:
at least two side-viewing optical assemblies, each having a different radial field of view.

10. The catheter of claim 9, wherein each side-viewing optical assembly is associated with at least one optical fiber that longitudinally spans the body of the catheter from its proximal end to the side-viewing optical assembly so that light transmitted from the proximal end of the catheter can be directed to a blood vessel wall and light received from a blood vessel wall can be transmitted out of the catheter for analysis.

11. The catheter of claim 9, wherein the side-viewing optics comprise:
four side-viewing optical assemblies, each radially separated from adjacent assemblies by 90-deg or approximately 90-deg and each having an at least substantially non-overlapping radial field of view with the other.

12. The catheter of claim 11, wherein each side-viewing optical assembly is associated with at least one optical fiber that longitudinally spans the body of the catheter from its proximal end to the side-viewing optical assembly so that light transmitted from the proximal end of the catheter can be directed to a blood vessel wall and light received from a blood vessel wall can be transmitted out of the catheter for analysis.
